(19) United States
(12) Patent Application Publication
(10) Pub. No.: US 2022/0047786 A1
(43) Pub. Date: Feb. 17, 2022

(54) BREAST MILK PUMP

(71) Applicant: Annabella Tech Ltd., Kfar Saba (IL)

(72) Inventors: Andres Wainstein, Ramat Gan (IL); Gustavo Turkieltaub, Tel Aviv (IL); Adi Shfaram, Herzliya (IL)

(73) Assignee: Annabella Tech Ltd., Kfar Saba (IL)

(21) Appl. No.: 16/990,111

(22) Filed: Aug. 11, 2020

(51) Int. Cl. A61M 1/06; A61M 1/00

(52) U.S. Cl. CPC A61M 1/064; A61M 2210/1007; A61M 1/0009

(57) ABSTRACT

A breast milk pump includes a breast interface member which has an inner cavity through which milk can flow, and a negative pressure assembly in fluid communication with one side of a diaphragm. An opposite side of the diaphragm is in fluid communication with the inner cavity. The diaphragm separates the inner cavity from the negative pressure assembly. A suckling assembly includes a tongue member and an actuator coupled to the tongue member for causing movement of the tongue member. A tongue position adjustment assembly is coupled to the suckling assembly and operative to cause displacement of the suckling assembly with respect to an inner surface of the inner cavity.

### FIELD OF THE INVENTION

The present invention relates generally to a breast milk pump and method for collecting milk from a breast of a nursing mother, and particularly to a breast milk pump system with user friendly features.

### BACKGROUND OF THE INVENTION

Breastfeeding is the best source of nutrition for a baby and provides health benefits to the nursing mother. Sometimes a nursing mother needs to use a breast pump to collect milk. A variety of breast pumps are available, such as hand-operated pumps and electric pumps. The electric pumps may be battery and/or AC powered.

Even though there are many breast pumps available, the need still exists for an improved user-friendly breast pump.

### SUMMARY OF THE INVENTION

The present invention seeks to provide an improved breast milk pump system with user friendly features, as is described more in detail hereinbelow.

There is provided in accordance with a non-limiting embodiment of the invention a breast milk pump including a breast interface member which has an inner cavity through which milk can flow, a negative pressure assembly in fluid communication with one side of a diaphragm, an opposite side of the diaphragm being in fluid communication with the inner cavity, the diaphragm separating the inner cavity from the negative pressure assembly, a suckling assembly including a tongue member and an actuator coupled to the tongue member for causing movement of the tongue member, and a tongue position adjustment assembly coupled to the suckling assembly and operative to cause (e.g., relatively large and relatively small) displacement of the suckling assembly with respect to an inner surface of the inner cavity. The tongue member is the element that touches the breast and/or nipple to suckle milk.

The suckling assembly may be rotatingly mounted on a tongue lever arm at one side of a pivot and the tongue position adjustment assembly may be coupled to the tongue lever arm at an opposite side of the pivot.

The tongue position adjustment assembly may include a wheel with a spiral channel formed therein, and the tongue lever arm may be coupled to the tongue position adjustment assembly by means of a pin which may be received in the spiral channel. When the pin may be at a radially outermost end of the spiral channel, the suckling assembly may be at a position closest to the inner surface of the inner cavity and when the pin may be at a radially innermost end of the spiral channel, the suckling assembly may be at a position furthest from the inner surface of the inner cavity.

At one portion of the spiral channel, relatively small rotational movement of the wheel causes relatively larger displacement of the pin and relatively larger displacement of the suckling assembly and at another portion of the spiral channel, relatively large rotational movement of the wheel causes relatively smaller displacement of the pin and relatively smaller displacement of the suckling assembly. Any force (vertical or any orientation) on the suckling assembly may be countered by the pin abutting against an inner surface of the spiral channel.

The inner cavity may extend from a funnel end to an intermediate volume, and one portion of the intermediate volume may be in fluid communication with an air conduit and another portion of the intermediate volume may be in fluid communication with a milk collection vessel. A one-way valve may be located between the intermediate volume and the milk collection vessel.

A liquid barrier may be positioned near the air conduit, the barrier being slanted so that any milk sucked through the inner cavity to the intermediate volume cannot flow past the barrier and is forced to flow towards the milk collection vessel, but air or any gas can flow past the barrier towards the diaphragm.

The inner cavity may extend to an intermediate volume which may be in fluid communication with a milk collection vessel, and a flow of all milk that enters the inner cavity may be through the intermediate volume and into the milk collection vessel and the flow may be completely separated from the negative pressure assembly.

The negative pressure assembly may include a receiving member in which the diaphragm may be received, the receiving member including a first electrical connector, and a hinged member pivotally coupled to the receiving member at a hinge, the hinged member including a second electrical connector arranged for electrical connection with the first electrical connector.

The negative pressure assembly may include a flange, through which extends a negative pressure conduit and electrical wiring.

### BRIEF DESCRIPTION OF THE DRAWINGS

The present invention will be understood and appreciated more fully from the following detailed description, taken in conjunction with the drawings in which:

FIG. 1 and FIG. 1A are simplified side view and enlarged view illustrations, respectively, of a breast milk pump, in accordance with a non-limiting embodiment of the invention.

FIG. 2 is a more detailed illustration of a suckling assembly of the breast milk pump, in accordance with a non-limiting embodiment of the invention, including a tongue member, tongue actuator, tongue lever arm and tongue position adjustment assembly.

FIGS. 3 and 4 are simplified side view and perspective view illustrations, respectively, of the tongue actuator, tongue lever arm and tongue position adjustment assembly.

FIG. 5 is a simplified side view illustration of a negative pressure assembly of the breast milk pump, in accordance with a non-limiting embodiment of the invention.

FIG. 6 is a simplified perspective view illustration of the negative pressure assembly, showing a diaphragm, hinged connector and electrical connector, in accordance with a non-limiting embodiment of the invention.

### DETAILED DESCRIPTION OF EMBODIMENTS

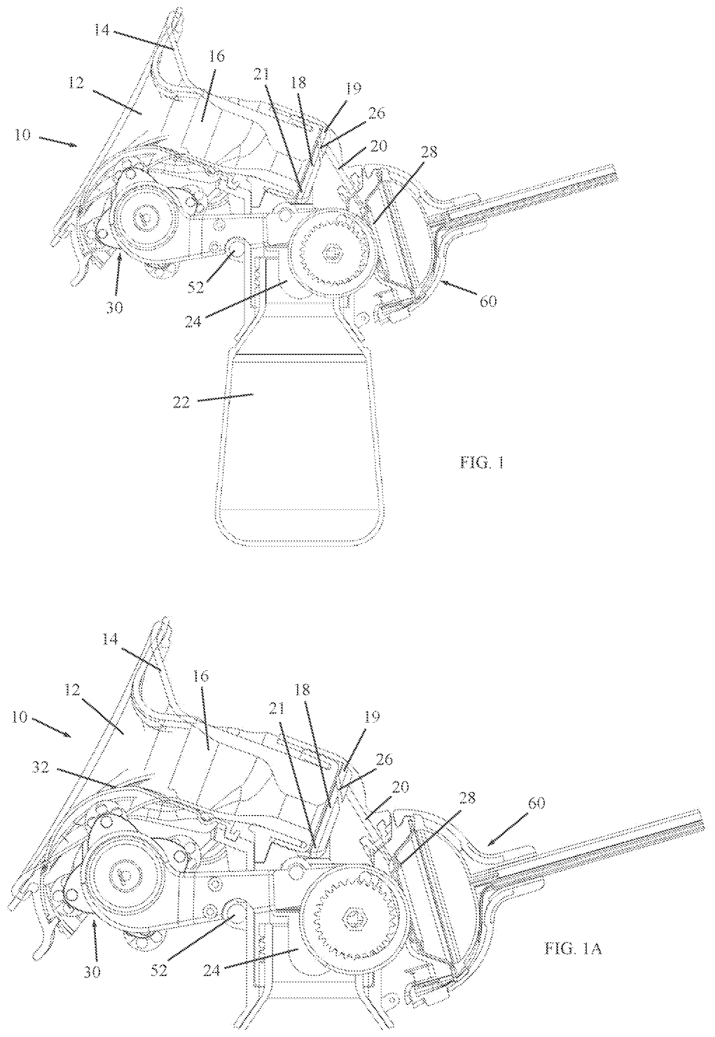

Reference is now made to FIGS. 1 and 1A, which illustrate a breast milk pump 10, constructed and operative in accordance with a non-limiting embodiment of the present invention.

The pump 10 includes a breast interface member 12, which may have a funnel end 14 (which may or may not be flared) for fitting over a breast. The breast interface member 12 has an inner cavity 16 through which milk can flow. The breast interface member 12 may be made of one piece or multiple pieces assembled together. The inner cavity 16 extends from the funnel end 14 to an intermediate volume 18. An upper portion 19 (“upper” in the sense of the drawing) of the intermediate volume 18 is in fluid communication with an air conduit 20, whereas a lower portion 21 (“lower” in the sense of the drawing) of the intermediate volume 18 is in fluid communication with a milk collection vessel 22. A one-way valve 24 may be located between the lower portion 21 of the intermediate volume 18 and milk collection vessel 22. The one-way valve 24 may permit milk to flow from the lower portion 21 of the intermediate volume 18 to the milk collection vessel 22, but blocks flow out of vessel 22.

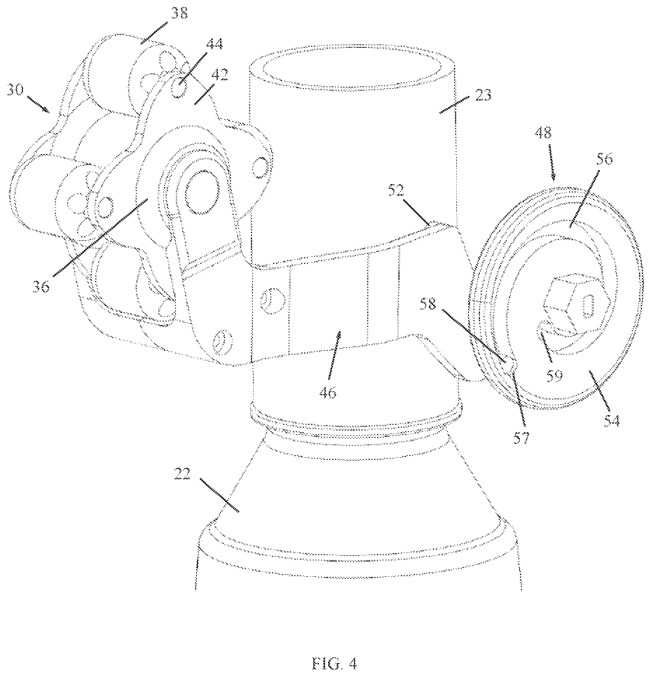

The milk collection vessel 22 may be sealingly secured (such as by threaded connection and O-ring) to a port housing 23 (also seen in FIGS. 3 and 4), which is part of said breast interface member 12.

A liquid barrier 26 may be positioned just below air conduit 20. Barrier 26 may be slanted so that any milk sucked through inner cavity 16 to intermediate volume 18 cannot flow past barrier 26 and is forced to flow only downwards (typically by the force of gravity) to the lower portion 21 of intermediate volume 18 and to milk collection vessel 22. Barrier 26 does not completely block the upper portion 19 of the intermediate volume 18 so that air or any other gas (not liquid) can flow past the upper end of barrier 26 and flow through air conduit 20 towards a diaphragm 28.

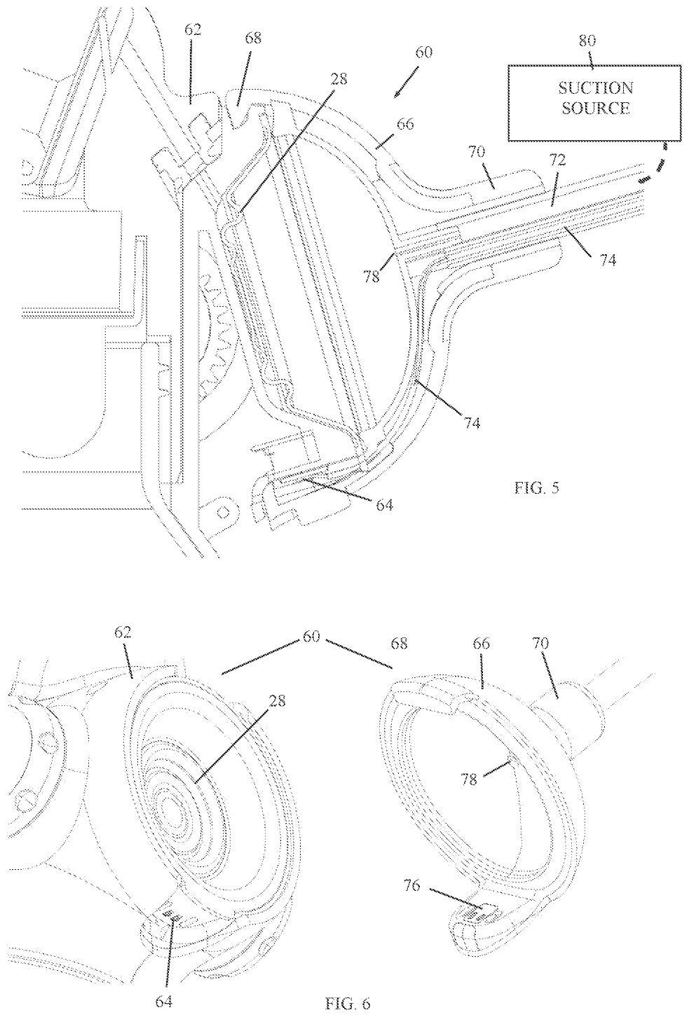

Diaphragm 28 is part of a negative pressure assembly of the breast milk pump, which is described below with referenced to FIGS. 5 and 6. Diaphragm 28 moves in response to application of negative pressure on the side opposite to air conduit 20. This movement of diaphragm 28 creates negative pressure in the air conduit 20, intermediate volume 18 and inner cavity 16, when breast interface member 12 is pressed against the breast of the mother. The negative pressure causes the milk to flow into vessel 22, whereas any air trapped in the volume from inner cavity 16, intermediate volume 18 and air conduit 20 flows towards diaphragm 28 (its left side in the sense of the drawing).

Accordingly, all milk that enters inner cavity 16 of breast interface member 12 flows through intermediate volume 18 down to its lower portion 21, through the optional one-way valve 24 and into milk collection vessel 22. It is noted that there is a continuous downward flow of milk from breast interface member 12 to milk collection vessel 22 and the flow is completely separated from the negative pressure assembly of the breast milk pump.

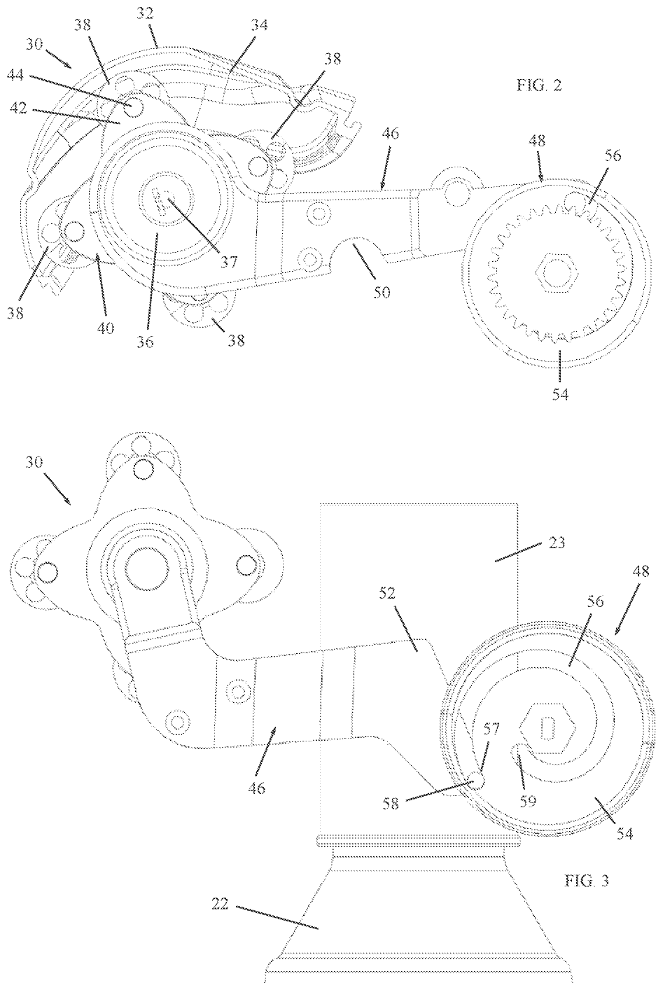

Reference is now made to FIG. 2, which illustrates a suckling assembly 30 of the breast milk pump, in accordance with a non-limiting embodiment of the invention.

The suckling assembly 30 may include a tongue member 32, which may be made of an elastomeric material, and may be mounted on a carrier member 34 (tongue member 32 and carrier member 34 may be one and the same). Carrier member 34 is in contact with a roller assembly, which includes one or more rollers 38 (in the non-limiting embodiment there are four rollers 38, but the invention is not limited to this number). The rollers 38 may be mounted on a rotating member 40, which may include one or more arms 42 to which each of the one or more rollers 38 is pivotally attached at a pivot 44 (such as a pin or rivet). A tongue actuator 36, such as a motor which may be controlled by control electronics with feedback sensors and components, may be operationally coupled to the roller assembly to rotate rotating member 40. In a preferred embodiment, tongue actuator 36 is mounted at the axle 37 of rotating member 40 (the axle 37 of the actuator 36 is the axle of the rotating member 40), which saves on space and obviates any need for linkage or gearing to rotating member 40.

In one aspect of the invention, the negative pressure applied to the diaphragm 28 is synchronized with the suckling action. This maximizes the amount of milk drawn by the suckling action from the mother's breast. One way of achieving synchronization is with software in the vacuum unit (negative pressure source). Another way is to use the control electronics and optional feedback sensors of the tongue actuator 36 to provide the synchronization.

As rotating member 40 rotates, the rollers 38 sequentially move the carrier member 34, and along with it, the tongue member 32, so that the tongue member 32 moves in a reciprocating motion towards the front (funnel end 14) of breast interface member 12 (FIG. 1) and away from the funnel end 14 of breast interface member 12, plus the tongue member 32 moves upwards (when the roller is at its maximum upward motion; the 12 o'clock position in the illustrated embodiment) and downwards (between adjacent rollers). This mimics the action of a baby's tongue when suckling milk from the mother's breast. The upward movement of the rollers presses the tongue member against the nipple and the rearward movement provides the suckling action. The vacuum provided by the negative pressure assembly draws the sucked milk eventually to the milk collection vessel.

The anatomical geometry of the mother's breast varies from one mother to another. Accordingly, the invention provides the ability to adjust the vertical position (as always, “vertical” in the sense of the drawings) of the tongue member 32 so that each mother can individually place the tongue member at the optimal position for suckling milk from the breast. In addition, the invention enables coarse and fine adjustment: coarse adjustment allows the user to rapidly move the tongue member in a relatively large movement, such as for initial adjustment, whereas fine adjustment allows the user to fine-tune the final desired position of the tongue member in relatively small movements. At all times, the adjustment is steady and lockable in place at a desired position.

In accordance with a non-limiting embodiment of the invention, this is achieved by means of a tongue position adjustment assembly 48, as is now described.

The suckling assembly 30 (including tongue member 32, carrier member 34, one or more rollers 38, rotating member 40, and tongue actuator 36) may be rotatingly mounted (at axle 37) on a tongue lever arm 46 at one side of a pivot socket 50 formed on arm 46. The tongue position adjustment assembly 48 may be coupled to arm 46 at an opposite side of pivot socket 50. Pivot socket 50 rests on and pivots about a pivot 52 (FIG. 1). Alternatively, as seen in FIGS. 3 and 4, pivot 52 may be at a different portion of arm 46 so that arm 46 pivots on port housing 23.

Reference is now made to FIGS. 3 and 4. The tongue position adjustment assembly 48 includes a wheel 54 with a spiral channel 56 formed therein. The spiral channel 56 may be, without limitation, a logarithmic spiral. The tongue lever arm 46 is coupled to tongue position adjustment assembly 48 by means of a pin 58 which is received in spiral channel 56 (or any other suitable member that can move in spiral channel 56, such as a protrusion, lug, rivet and many more, and the terms are used interchangeably).

In FIG. 3, pin 58 is shown at the radially outermost end 57 of spiral channel 56. This is lowest position of pin 58. Since arm 46 pivots about pivot 52, when pin 58 is at its lowest position, suckling assembly 30 is at its highest position (that is, closest to the inner surface of inner cavity 16). Conversely, when pin 58 is at the radially innermost end 59 of spiral channel 56, pin 58 is at its highest position and suckling assembly 30 is at its lowest position (that is, furthest from the inner surface of inner cavity 16).

The spiral shape of spiral channel 56 provides coarse and fine adjustment of the position of suckling assembly 30 (and tongue member 32, not shown) as is now explained.

Starting at the radially innermost end 59 of spiral channel 56, relatively small rotational movement of wheel 54 (counterclockwise in the sense of the drawing) causes relatively larger vertical displacement of pin 58 and thus relatively larger vertical displacement of suckling assembly 30. This provides coarse adjustment. Once pin 58 has reached approached the radially outer portion of spiral channel 56 (starting about 11 o'clock in the sense of FIG. 3), the relatively large movement of pin 58 towards the radially outermost end 57 of spiral channel 56 cause relatively smaller vertical displacement of pin 58 and thus relatively smaller vertical displacement of suckling assembly 30. This provides fine adjustment. Wheel 54 is thus an adjustment knob or handle, the terms being used interchangeably. There may be left and right wheels 54 on opposite sides of the breast milk pump. Accordingly, tongue position adjustment assembly 48 may be coupled to suckling assembly 30 with left and right adjustment handles 54.

In addition, the spiral shape of spiral channel 56 provides a locking effect that locks the pin 58 and thus the suckling assembly 30 at a desired position. The reason is that any force (downward or upward or other directions) on suckling assembly 30 is countered by pin 58 abutting against the inner surface of the spiral channel 56, thereby preventing any movement (downward or upward or other directions) of suckling assembly 30.

The user may adjust the relative position of pin 58 in spiral channel 56 by rotating wheel 54. The turning of wheel 54 may be direct, or alternatively, as seen in FIG. 2, may be done through a geared connection.

It is noted that tongue position adjustment assembly 48 may be coupled to suckling assembly 30 directly, instead of via tongue lever arm 46.

Reference is now made to FIGS. 5 and 6, which illustrate a negative pressure assembly 60 of the breast milk pump, in accordance with a non-limiting embodiment of the invention.

The negative pressure assembly 60 may include diaphragm 28, whose perimeter is affixed to a first member 62 of the body of the device. A first electrical connector 64 may be located at a lower portion (or other portion) of first member 62. A second member 66 may be pivotally coupled to first member 62 at a hinge 68; alternatively, second member 66 may be connectable to first member 62 in other ways, such as by snap or click connection, snug fit connection, threaded connection and others. The second member 66 may include a flange 70, such as a central, outwardly protruding flange 70, through which extends a negative pressure conduit 72 and electrical wiring 74. The electrical wiring 74 may be connected to a second electrical connector 76. Second member 66 may be formed with an aperture 78 which is fluidly connected to negative pressure conduit 72. As seen in FIG. 5, is fluidly connected to a negative pressure source 80 (e.g., a vacuum pump). When second member 66 is closed against first member 62 (there may be a seal such as an O-ring to seal second member 66 against first member 62), the negative pressure source 80 can provide the negative pressure required to draw the milk into the collection vessel, as described above.

The negative pressure assembly 60 thus provides both electrical and negative pressure connection in one assembly. Connection of first member 62 and second member 66 achieves negative pressure and electrical power connections.

What is claimed is:

1. A breast milk pump comprising:
a breast interface member which has an inner cavity through which milk can flow;
a negative pressure assembly in fluid communication with one side of a diaphragm, an opposite side of said diaphragm being in fluid communication with said inner cavity, said diaphragm separating said inner cavity from said negative pressure assembly;
a suckling assembly comprising a tongue member and an actuator coupled to said tongue member for causing movement of said tongue member; and
a tongue position adjustment assembly coupled to said suckling assembly and operative to cause displacement of said suckling assembly with respect to an inner surface of said inner cavity.

2. The breast milk pump according to claim 1, wherein said tongue position adjustment assembly is operative to cause relatively large and relatively small displacement of said suckling assembly

3. The breast milk pump according to claim 1, wherein said suckling assembly is rotatingly mounted on a tongue lever arm at one side of a pivot and said tongue position adjustment assembly is coupled to said tongue lever arm at an opposite side of said pivot.

4. The breast milk pump according to claim 3, wherein said tongue position adjustment assembly includes a wheel with a spiral channel formed therein, and said tongue lever arm is coupled to said tongue position adjustment assembly by means of a pin which is received in said spiral channel.

5. The breast milk pump according to claim 4, wherein when said pin is at a radially outermost end of said spiral channel, said suckling assembly is at a position closest to the inner surface of said inner cavity and when said pin is at a radially innermost end of said spiral channel, said suckling assembly is at a position furthest from the inner surface of said inner cavity.

6. The breast milk pump according to claim 4, wherein at one portion of said spiral channel, relatively small rotational movement of said wheel causes relatively larger displacement of said pin and relatively larger displacement of said suckling assembly and at another portion of said spiral channel, relatively large rotational movement of said wheel causes relatively smaller displacement of said pin and relatively smaller displacement of said suckling assembly.

7. The breast milk pump according to claim 3, wherein any force on said suckling assembly is countered by said pin abutting against an inner surface of said spiral channel.

8. The breast milk pump according to claim 1, wherein said tongue position adjustment assembly is coupled to said suckling assembly with left and right adjustment handles.

9. The breast milk pump according to claim 1, wherein said inner cavity extends from a funnel end to an intermediate volume, and a portion of said intermediate volume is in fluid communication with a milk collection vessel.

10. The breast milk pump according to claim 1, wherein said inner cavity extends from a funnel end to an intermediate volume, and one portion of said intermediate volume is in fluid communication with an air conduit.

11. The breast milk pump according to claim 9, and comprising a one-way valve located between said intermediate volume and said milk collection vessel.

12. The breast milk pump according to claim 9, and comprising a liquid barrier positioned near said air conduit, said barrier being slanted so that any milk sucked through said inner cavity to said intermediate volume cannot flow past said barrier and is forced to flow towards said milk collection vessel, but air or any gas can flow past said barrier towards said diaphragm.

13. The breast milk pump according to claim 1, wherein a portion of said breast interface member is sealingly secured to a milk collection vessel.

14. The breast milk pump according to claim 1, wherein said negative pressure assembly comprises a first member in which said diaphragm is received, said first member comprising a first electrical connector, and a second member connectable to said first member, said second member comprising a second electrical connector arranged for electrical connection with said first electrical connector and a negative pressure conduit and electrical wiring for respectively supplying negative pressure and electrical power, such that connection of said first member and said second member achieves negative pressure and electrical power connections.

15. The breast milk pump according to claim 1, wherein negative pressure applied to said diaphragm is synchronized with suckling action of said suckling assembly.
